(19) United States
(12) Patent Application Publication
(10) Pub. No.: US 2003/0176382 A1
(43) Pub. Date: Sep. 18, 2003

(54) Treatment of hookworm infection

(76) Inventor: David Idris Pritchard, Nottingham (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/220,083

(22) PCT Filed: Feb. 26, 2001
(86) PCT No.: PCT/GB01/00819

(30) Foreign Application Priority Data
Feb. 25, 2000 (GB) 0004533.6

(51) Int. Cl. A61K 48/00; A61K 39/002; C07K 16/40

(52) U.S. Cl. 514/44; 424/265.1; 530/388.26

(57) ABSTRACT

Vaccine compositions for the treatment of hookworm infections comprise antigenic fragments of aspartyl proteinases obtainable from Necator americanus.

### FIELD OF THE INVENTION

This invention relates to the production of vaccine compositions to treat parasitic infection, in particular to treat infection of the hookworm Necator americanus.

### BACKGROUND OF THE INVENTION

The human hookworm Necator americanus is a human pathogen that invades the body by penetrating the skin, and causes debilitating iron deficiency anaemia at low infection intensity.

Treating infection using pharmaceuticals can be carried out but the effect is often transient, and the treatment is costly. Hookworm vaccines have been used successfully to control the pathology associated with canine infections. However, protection in this case was induced by exposure to live γ-radiation-attenuated infective larvae, and this treatment is unlikely to be acceptable for human use.

Matthews, Z Parasitenkd, 1982; 68: 81-86, discloses that cellular destruction of the skin during larval penetration through the epidermis is effected by an undefined enzymatic process. It was shown subsequently that serine, and possibly cysteinyl, proteinases were responsible for skin penetration. However, the precise role for each of these proteinases was not defined.

Brown et al, Am. J. Trop. Med. Hyg., 1999; 60(5): 840-847, identifies aspartyl proteinase activity to be important for larval stage skin penetration. No specific aspartyl proteinase is identified. Treatment to prevent skin penetration is proposed using general aspartyl proteinase inhibitors.

### SUMMARY OF THE INVENTION

The present invention is based on the realisation that the aspartyl proteinase of Necator americanus is a viable target for vaccine therapy.

Not only may aspartyl proteinases be important for larval stage hookworm skin penetration, but it is now appreciated that they may be important in the maintenance of the mature parasite life-cycle. This is based on the finding that adult parasites appear to depend predominantly on aspartyl proteinase activity to digest host haemoglobin and fibrinogen, which may be important to the maintenance of a haematophagous existence in the gut.

According to a first aspect of the invention, a vaccine composition comprises an aspartyl proteinase obtainable from the hookworm Necator americanus, or an antigenic fragment thereof.

According to a second aspect of the invention, a vaccine composition comprises a polynucleotide that encodes an aspartyl proteinase obtainable from the hookworm Necator americanus, or an antigenic fragment thereof.

According to a third aspect of the invention, an antibody is raised against an aspartyl proteinase, as defined above, and may be used in therapy or diagnosis.

According to a fourth aspect of the invention, an aspartyl proteinase comprises the amino acid sequence identified herein as SEQ ID NO. 6. This aspartyl proteinase is found only in the adult hookworm Necator americanus, and is structurally different from that found in larval forms.

In contrast to the prior art, the present invention provides means to treat an existing infection or to prevent infection. It was not at all apparent, until now, that an effective vaccine could be produced using an aspartyl proteinase as the antigenic fragment, and that this would be effective against hookworm infection. The identification of structurally different aspartyl proteinases is an important aspect in the development of the vaccine compositions, as is the finding that these are structurally different from the human aspartyl proteinases. It is particularly surprising that the adult hookworm contains structurally different aspartyl proteinases to that of the larval hookworm.

### DESCRIPTION OF THE INVENTION

The present invention provides treatments for parasitic infection, in particular from infection by human hookworm, e.g. Necator americanus. However, it is not intended to restrict the treatments to infections of a human host, and the present invention extends to veterinary treatment of animal infections, for example, by the related canine hookworm Ancylostoma caninum, or the sheep hookworm Haemonchus contortus.

Specific aspartyl proteinases are identified herein on the basis of polynucleotide and amino acid sequences (identified herein as SEQ ID NOS. 2, 3, 4 and 6). Homologues to these sequences, with at least 60%, preferably at least 80% or 90%, sequence identity or similarity (measured across the complete sequence) are also within the scope of the invention.

The terms “similarity” and “identity” are known in the art. The use of the term “identity” refers to a sequence comparison based on identical matches between correspondingly identical positions in the sequences being compared. The term “similarity” refers to a comparison between amino acid sequences, and takes into account not only identical amino acids in corresponding positions, but also functionally similar amino acids in corresponding positions. Thus, similarity between polypeptide sequences indicates functional similarity, in addition to sequence similarity.

Levels of identity between gene sequences and levels of identity or similarity between amino acid sequences can be calculated using known methods. In relation to the present invention, publicly available computer-based methods for determining identity and similarity include the BLASTP, BLASTN and FASTA (Atschul et al., J. Molec. Biol., 1990; 215:403-410), the BLASTX program available from NCBI, and the Gap program from Genetics Computer Group, Madison Wis. The levels of similarity and identity referred to herein, are calculated with reference to the Gap program, with a Gap penalty of 12 and a Gap length penalty of 4 for determining the amino acid sequence comparisons, and a Gap penalty of 50 and a Gap length penalty of 3 for the polynucleotide sequence comparisons.

The aspartyl proteinases according to the invention may be purified and isolated by methods known in the art. In particular, having identified the gene sequence or the N-terminal sequence, it will be possible to use recombinant techniques to express the genes in a suitable host.

Active fragments of the proteins and polynucleotides are those that retain the biological function of the protein or polynucleotide. For example, when used as part of the vaccine to elicit an immune response, the fragment will be of sufficient size, such that antibodies generated in response to the fragment will be specific for that aspartyl proteinase and will not, for example, cross-react with the natural aspartyl proteinases of the patient. Typically, the fragment will be at least 30 nucleotides (10 amino acids) in size, preferably 60 nucleotides (20 amino acids) and most preferably greater than 90 nucleotides (30 amino acids) in size.

It should also be understood that the invention encompasses modifications made to the proteins and polynucleotides identified herein which do not significantly alter the biological function. It will be apparent to the skilled person that the degeneracy of the genetic code can result in polynucleotides with minor base changes from those specified herein, but which nevertheless encode the same proteins. Complementary polynucleotides are also within the invention. Conservative replacements at the amino acid level are also envisaged, i.e. different acidic or basic amino acids may be substituted without substantial loss of function.

The preparation of vaccines based on the aspartyl proteinases will be apparent to those skilled in the art. Vaccine compositions can be formulated with suitable carriers or adjuvants, e.g. alum, as necessary or desired, to provide effective immunisation against infection.

It is preferred that the vaccines are prepared in order to elicit a T-helper type-2 cell response. The adjuvant may therefore comprise components that influence this, and it may be preferable not to include adjuvants comprising bacterial components which induce T-helper type-1 cell responses.

More generally, and as is well known to those skilled in the art, a suitable amount of an active component of the invention can be selected, for therapeutic use, as can suitable carriers or excipients, and routes of administration. These factors would be chosen or determined according to known criteria such as the nature/severity of the condition to be treated, the type and/or health of the subject etc.

The vaccine may comprise an antigenic fragment of an aspartyl proteinase characterised as present in the larval stage, or alternatively, present in the adult stage. In a preferred embodiment, the vaccine composition comprises a combination of an antigenic fragment derived from a larval stage aspartyl proteinase and an antigenic fragment derived from an adult stage aspartyl proteinase. This offers maximum protection as it targets separate stages of hookworm infection.

In a further preferred embodiment, the aspartyl proteinase from which the vaccine may be prepared, is encoded by the DNA sequence defined as SEQ ID NO. 1, or SEQ ID NO. 5, or a homologue thereof with at least 60% sequence identity, preferably 80%, and most preferably 95% sequence identity.

The vaccine may also be derived from an aspartyl proteinase characterised as comprising an amino acid sequence shown as SEQ ID NO. 3 or SEQ ID NO. 4.

The vaccine may comprise alternatively a genetic construct that encodes an aspartyl proteinase, or a fragment thereof. In this embodiment, it may be necessary to prepare the construct to include appropriate regulatory factors, e.g. promoters, in addition to the polynucleotide that encodes the proteinase. Suitable components, including suitable vectors, will be apparent to the skilled person.

The invention will now be further described by way of example only with reference to aspartyl proteinases isolated from N. americanus.

### EXAMPLE

### Preparation of N. Americanus Larval Secretions

Infective larvae were cultured from faecal material as described by Kumar and Pritchard, Int. J. Parasitol, 1992; 22:563-572. Briefly, faecal material obtained from hamsters infected with N. americanus was mixed with activated charcoal, 1% (w/v) amphotericin B and water to form a smooth paste which was applied to the upper half of a 5×30 cm strip of filter paper. These strips were then suspended in a large glass chromatography tank containing approximately 750 ml of distilled water. The tanks were sealed and incubated at 28° C. for 10 days, after which the filter paper strips were carefully removed and discarded. The water containing the larvae was transferred to a measuring cylinder and the larvae allowed to sediment for two hours. After this period the water was aspirated off and the larvae washed twice to remove any faecal contamination. Finally, washed larvae were re-suspended in approximately 20 ml of storage buffer (50 mM Na₂HPO₄, 70 mM NaCl, 15 mM KH₃PO₄, pH 7.4). Larvae were stored in the dark at room temperature until required, or for a maximum period of one month.

Excretory-secretory (ES) products were collected as described by Kumar and Pritchard (1992), supra. Freshly collected, ensheaved larvae were re-suspended in larval storage buffer and exsheaved by bubbling carbon dioxide through the suspension for two hours at room temperature. Exsheaved larvae were allowed to settle and then washed extensively with RPMI 1640 containing 100 i.u./ml penicillin, 100 μg/ml streptomycin and 1% amphotericin B under sterile conditions. Following this sterilisation period the larvae were cultured in RPMI 1640 containing the above additives for 72 hours at 37° C., changing the culture medium every 24 hours. ES products collected over the 72 hour period were pooled, dialysed against distilled water, lyophilized and stored at −20° C. until required.

### Enzyme Purification

Substrate SDS-PAGE was carried out using a method modified from Pritchard et al, Parasitology Today, 1990; 6: 154-156. 12% (w/v) SDS-PAGE gels were prepared with the inclusion of 0.1% (w/v) haemoglobin in the resolving gel. 10 μg of the ES products was mixed with an equal volume of non-reducing sample buffer (0.5M Tris, pH 6.8, 5% SDS (w/v), 20% glycerol (w/v), 0.01% bromophenol) and incubated under 37° C. for 30 minutes. The sample was then applied to the gel which was then electrophoresed at a constant current of 20 mA. Following electrophoresis, the gels were washed in 2.5% Triton X-100 for one hour at room temperature to renature the enzymes. The gels were then washed in water for 30 minutes, cut into individual strips and incubated for 48 hours at 37° C. in 0.1 M sodium phosphate buffer pH 6.5. Proteinase activity was detected by staining gels with Coomassie brilliant blue R250.

The gels revealed three proteinase products at 31 kD, 33 kD and 35 kD.

Larval aspartyl proteinase was purified from the ES products using pepsatin A agarose (Sigma). A 5 ml pepsatin A agarose column was equilibrated with 50 mM sodium acetate pH 5.5. The ES products in 50 mM sodium acetate pH 5.5 were applied to the column at a flow rate of 0.2 ml/min. The column was washed sequentially with 10 ml, 50 mM sodium acetate pH 5.5 followed by 10 ml, 50 mM sodium acetate, 0.5 M sodium chloride pH 5.5. Bound protein was eluted from the column with 15 ml, 500 μM pepsatin A dissolved in 50 mM sodium acetate pH 5.5. One ml fractions were collected and analysed for protein content and proteolytic activity using FITC-labelled casein. Fractions eluted from the column containing 500 μM pepsatin A were dialysed against distilled water prior to analysis for proteolytic activity.

The aspartyl proteinases present in the purified fractions were sequenced to obtain information on their amino acid and nucleic acid structure. The DNA sequence for one of the larval aspartyl proteinases is shown as SEQ ID NO. 1 and the amino acid sequence is shown as SEQ ID NO. 2. N-terminal sequencing was carried out for two other larval aspartyl proteinases, and the sequences are shown as SEQ ID NOS. 3 and 4.

The measurement of proteinase activity, using FITC-casein as the substrate, revealed that the activity was optimal at pH 6.5. At pH 6.5, proteinase activity was also shown to be inhibited by pepsatin A.

An aspartyl proteinase was also purified from the adult hookworm using techniques similar to those described. This proteinase had an amino acid and nucleic acid sequence significantly different to those obtained from the larval hookworm. The nucleic acid sequence is shown as SEQ ID NO. 5 and the amino acid sequence is shown as SEQ ID NO. 6.

The aspartyl proteinase obtained from the adult form was tested in assays to determine its substrate specificity. It was found that the proteinase cleaved the synthetic peptide substrate ALERTFLSFPT (SEQ ID NO. 7). This synthetic substrate mimics the site at which initial cleavage of haemaglobin by P. falciparum aspartic proteinases is known to occur. Adult aspartyl proteinase may therefore be important in the digestion of host haemoglobin and fibrinogen and may therefore be an important factor in anti-coagulation, maintaining the hookworm in the host.

1. A vaccine composition comprising an aspartyl proteinase obtainable from the hookworm Necator americanus or an antigenic fragment thereof.

2. A composition according to claim 1, wherein the proteinase or fragment comprises part or all of any of the amino acid sequences defined herein as SEQ ID NOS. 2, 3, 4 and 6, or a homologue thereof with at least 60% sequence similarity.

3. A composition according to claim 1 or claim 2, wherein the fragment is at least 30 amino acids.

4. A composition according to any preceding claim, comprising both adult and larval aspartyl proteinases or antigenic fragments thereof.

5. A vaccine composition comprising a polynucleotide that encodes an aspartyl proteinase obtainable from the hookworm Necator americanus or an antigenic fragment thereof.

6. A composition according to claim 5, wherein the polynucleotide comprises SEQ ID NO. 1 or SEQ ID NO. 5, or a homologue thereof with at least 60% sequence identity.

7. A composition according to claim 5 or claim 6, comprising polynucleotides encoding each of adult and larval aspartyl proteinases or antigenic fragments thereof.

8. Use of an aspartyl proteinase as defined in any of claims 1 to 4, or a polynucleotide as defined in any of claims 5 to 7, in the manufacture of a vaccine composition for the treatment of a hookworm infection.

9. Use according to claim 8, wherein the infection is a Necator americanus infection.

10. Use according to claim 8, wherein the infection is an Ancylostoma caninum infection.

11. Use according to claims 8, wherein the infection is an Haemonchus contortus infection.

12. An antibody raised against an aspartyl proteinase as defined in any of claims 1 to 3.

13. An aspartyl proteinase obtainable from Necator americanus, encoded by a gene comprising the polynucleotide identified herein as SEQ ID NO. 5.

14. An aspartyl proteinase according to claim 13, for therapeutic use.


SEQUENCE LISTING (7 sequences)

SEQ ID NO: 1 - DNA, 1341 bp, Necator americanus
  CDS (1)..(1341)
atggctcgacttgtattcctactcgtactatgtactctggctgcagcaagcgttcatcgacgactctttcatcaagctcgtcgtcatgtgacatcggtatcgctttcgcgtcagccaacacttcgtgaacgactgatcgcaagtggcagttgggaggattaccagaaacaacgctaccattatcgaaagaaaattctagcaaaatatgctgctaacaaagcgtcaaagttacaatctgcaaacgagatcgatgaattgctccggaactatatggatgcacaatactatggtgtcatccaaattgggactccagctcagaatttcactgtgatcttcgacacgggttcctcaaatctatgggtaccgtcaagaaagtgtccattctatgacattgcatgtatgcttcatcatcgttatgactccggagcctcgtcaacctacaaggaagatgggcgcaagatggctattcagtatggaactggatctatgaaaggattcatttctaaggatattgtttgtattgctggaatttgcgctgaagaacaacctttcgcggaggctacaagtgaacctggtcttacatttatcgctgctaagtttgatggaatccttggaatggcattcccggaaattgctgttctcggtgtaactcctgtcttccatacgttcattgaacagaagaaagttcctagccctgtgtttgctttctggctgaataggaatccagagtcggaaattggaggagagattacctttggtggtgtggatacccgacgttatgttgaaccaattacatggacaccagtgacacgtcgtggatattggcaattcaaaatggatatggtacaaggtggttcatcgtccattgcgtgtccgaatggatgccaagctatcgctgatactggcacttctcttattgctggaccgaaggcacaggttgaggcaatccagaaatatatcggagcagagccgcttatgaaaggagaatacatgattccttgcgacaaagtaccatcccttcctgatgtttcgttcatcatcgatggcaagacgtttacactcaaaggggaagattacgttctaaccgtgaaagccgctggtaaatcaatctgtttgtctggcttcatgggaatggacttcccagagaagatcggcgaattgtggatccttggagatgttttcattggaaaatactacaccgtcttcgatgttggtcaggcacgtgttggatttgctcaagcaaagtcagaagatggattccctgttggcacccccgttcgaacattcagacagcttcaggaagacagcgatagcgacgaggacgatgtatttactttttaa

SEQ ID NO: 2 - protein, 446 aa, Necator americanus
MARLVFLLVLCTLAAASVHRRLFHQARRHVTSVSLSRQPTLRERLIASGSWEDYQKQRYHYRKKILAKYAANKASKLQSANEIDELLRNYMDAQYYGVIQIGTPAQNFTVIFDTGSSNLWVPSRKCPFYDIACMLHHRYDSGASSTYKEDGRKMAIQYGTGSMKGFISKDIVCIAGICAEEQPFAEATSEPGLTFIAAKFDGILGMAFPEIAVLGVTPVFHTFIEQKKVPSPVFAFWLNRNPESEIGGEITFGGVDTRRYVEPITWTPVTRRGYWQFKMDMVQGGSSSIACPNGCQAIADTGTSLIAGPKAQVEAIQKYIGAEPLMKGEYMIPCDKVPSLPDVSFIIDGKTFTLKGEDYVLTVKAAGKSICLSGFMGMDFPEKIGELWILGDVFIGKYYTVFDVGQARVGFAQAKSEDGFPVGTPVRTFRQLQEDSDSDEDDVFTF

SEQ ID NO: 3 - protein, 10 aa, Necator americanus
DVIPQVAHDY

SEQ ID NO: 4 - protein, 15 aa, Necator americanus
GNVVPQAVNDFTDVQ

SEQ ID NO: 5 - DNA, 1278 bp, Necator americanus
  CDS (1)..(1278)
atgcgttcgatactcgtgttggtggctctgatcggatgcattgctgcgggtgtatataaaatcccattgaaaagaatcactccgccgatgataaaaatgttgagagctggtacttgggaaacgtacgtagaaggaatgaggaagagacaattacagttactgaaggagcacaaggttcatatccaagatgtactcggctatgctaacatggagtacctcggcgaaattactattggaactcctcaacagaagtttctggtggttttggacactggctcctcgaatctgtgggtccctgatgattcatgctacaaggagaagagacctgatagatgtctagtatcaaactgtgatgctggactggtttgtcaagtcttctgtccagatcctaaatgctgtgaacatacgagagaattcaagcaagtaaacgcatgcaaagataagcatcgatttgatcaaaagaattccaacacttatgttaaaacaaacaaaacatgggcaatagcgtatggaactggagatgcgaggggattttttggaagagatacagtccgtttgggtgctgaaggaaaggatcagctcgttattaatgatacgtggttcggacaagcagagcatatagctgaatttttcagtaatactttccttgatggcattctcggactcgcttttcaagaactgtcagaaggaggcgtcgctcctccaataattcgtgccattgaccttggacttctcgatcaaccaatatttactgtctatttcgaaaatgtcggagacaaagaaggtgtttatggaggtgttttcacctggggtggtctcgatcccgatcattgcgaagatgaggtcacatatgaacagctaaccgaagcaacttactggcagtttagacttaaaggagtgtcgtctaagaacttctcgtcgacggctggttgggaagcaatatccgacactggtacctcgttaaatggagcccctagggggatactaagaagtattgcaagacagtataatggacagtacgtcgcatctcaaggtctctacgtcgtcgactgcagtaaaaatgtgaccgttgacgtgaccattggcgacagaaactacactatgactgcgaaaaatctcgtacttgaaatacaggctgatatatgtattatggcatttttcgaaatggacatgttcattggaccagcatggattcttggcgatccatttattcgagaatattgcaatattcatgacattgaaaagaagcggattggttttgcagctgtaaaacattga

SEQ ID NO: 6 - protein, 425 aa, Necator americanus
MRSILVLVALIGCIAAGVYKIPLKRITPPMIKMLRAGTWETYVEGMRKRQLQLLKEHKVHIQDVLGYANMEYLGEITIGTPQQKFLVVLDTGSSNLWVPDDSCYKEKRPDRCLVSNCDAGLVCQVFCPDPKCCEHTREFKQVNACKDKHRFDQKNSNTYVKTNKTWAIAYGTGDARGFFGRDTVRLGAEGKDQLVINDTWFGQAEHIAEFFSNTFLDGILGLAFQELSEGGVAPPIIRAIDLGLLDQPIFTVYFENVGDKEGVYGGVFTWGGLDPDHCEDEVTYEQLTEATYWQFRLKGVSSKNFSSTAGWEAISDTGTSLNGAPRGILRSIARQYNGQYVASQGLYVVDCSKNVTVDVTIGDRNYTMTAKNLVLEIQADICIMAFFEMDMFIGPAWILGDPFIREYCNIHDIEKKRIGFAAVKH

SEQ ID NO: 7 - protein, 11 aa, Artificial Sequence
  Synthetic peptide
ALERTFLSFPT